(19) United States
(12) Patent Application Publication
(10) Pub. No.: US 2015/0297807 A1
(43) Pub. Date: Oct. 22, 2015

(54) DEVICES AND METHODS FOR TREATING FISTULAS

(71) Applicant: VOLCANO CORPORATION, San Diego, CA (US)

(72) Inventors: Christopher Leblanc, San Diego, CA (US); Jeremy Stigall, San Diego, CA (US)

(21) Appl. No.: 14/610,315

(22) Filed: Jan. 30, 2015

Related U.S. Application Data
(60) Provisional application No. 61/933,408, filed on Jan. 30, 2014.

(51) Int. Cl. A61M 1/00; A61B 8/12; A61M 25/10; A61B 19/00

(52) U.S. Cl. CPC A61M 1/008; A61B 19/54; A61B 8/12; A61M 25/10; A61B 2019/5466; A61B 2019/5437; A61M 2025/1052

(57) ABSTRACT

The invention generally relates to treatment of fistulas and abscesses and specifically to an all-in-one treatment device. The invention provides an aspiration catheter with imaging capabilities that can also deploy a blocking device, thereby providing an all-in-one treatment device for treating a fistula or an abscess. The device is inserted into an abscess and its imaging capabilities can be used to guide the device to a treatment site. A clogging device can be deployed from the device to prevent draining into, or out of, the abscess. In certain aspects, the invention provides a device for aspirating an abscess or fistula that includes a catheter having an extended body with a lumen therein, an imaging device such as an ultrasonic transducer disposed at a distal portion of the catheter, and a clogging device deployable from the catheter.

### CROSS-REFERENCE TO RELATED APPLICATIONS

This application claims the benefit of, and priority to, U.S. Provisional Application Ser. No. 61/933,408, filed Jan. 30, 2014, the content of which is incorporated by reference herein in its entirety.

### FIELD OF THE INVENTION

The invention generally relates to treatment of fistulas and specifically to an all-in-one treatment device.

### BACKGROUND

A fistula is an abnormal connection between two tissue walls that can form in connection with an abscess. An abscess is a collection of pus or fluid in a cavity that may form in response to trauma such as a splinter, wound, or infection. Left untended, these lesions can lead to sepsis and other problems, particularly where for example a fistula connects to an anorectal abscess and seeps stool and pus. See, e.g., Juviler, 2008, Anorectal sepsis and fistula-in-ano, Surg Technol Int 17:139-149. A fistula and its related abscess must be treated to avoid the spread of infection.

Treatment of abscesses or fistulas generally involves drainage or blockage. For example, U.S. Pat. Nos. 6,428,041 and 7,981,041 relate to draining an abscess. Fistulas are more difficult as they typically should be both closed and drained. See, e.g., U.S. Pat. No. 8,177,809 to Mavani; U.S. Pub. 2013/0218201 to Obermiller; U.S. Pub. 2013/0158594 to Carrison; or U.S. Pub. 2011/0282334 to Groenhoff. Unfortunately, existing approaches require either blindly navigating the treatment device to the affected area or inserting a separate endoscope camera or imaging element, raising the risk of complications. Since navigation is difficult, blocking and draining a fistula is an imprecise operation with a risk of complication. Additionally, mis-directing an instrument can damage the tissue, making an abscess or fistula worse than it was to begin with.

### SUMMARY

The invention provides a treatment catheter with imaging capabilities that can also deploy a blocking device and be used to aspirate a fistula, thereby providing an all-in-one device for treating a fistula. The device is inserted into the fistula and its imaging capabilities can be used to guide the device to a treatment site. A clogging device can be deployed from the device to prevent drainage from an abscess into the fistula. Device markers can be used to determine a length of the fistula or a volume of fluid therein. The determined volume of fluid can then be aspirated from the fistula, thereby promoting rapid and thorough healing. Since the imaging device, the blocking device, and the aspiration lumen are all provided as part of a single device, all of the steps involved in aspiration of a fistula can be performed using a single device that only needs to be inserted into the patient a single time. Since all of the steps are performed with one insertion of a single device, the procedure is completed quickly and risks of contamination are minimized. Since imaging is provided by the device itself, navigation is easy and the fistula can be blocked and drained with accuracy and precision. Additionally, the imaging capabilities aids in avoiding mis-guiding the device and thus prevents unintended tissue damage.

Devices and methods of the invention may provide beneficial uses in the context of surgically formed fistulas such as arteriovenous (AV) fistulas created for hemodialysis. A device of the method may be used within an AV fistula to remove obstructions or thrombus and to obtain a desired patency. The aspiration lumen of the device may be used to drain unwanted material from an AV fistula with the clogging device useful for capturing the material to be drained.

In certain aspects, the invention provides a device for treating a fistula that includes a catheter for insertion into a fistula. An aspiration lumen extends through the length of the catheter. The device includes an imaging device such as an ultrasonic transducer disposed at a distal portion of the catheter and a clogging device deployable from the catheter. The device may include an inner shaft slidably disposed within a lumen, i.e., with the clogging device attached to the inner shaft when in an un-deployed state. The aspiration lumen may extend parallel to, or coaxially with, the lumen for the inner shaft. A suction device may be connected to the aspiration lumen. One or more conductors may extend from the imaging device along a length of the catheter to a connection plug at a proximal end of the catheter configured to be attached to an imaging system instrument. The device preferably includes at least one radiopaque marker near a distal portion of the catheter and at least one inked marker.

In some embodiments, the clogging device is a collapsible hoop that suspends a web to clog the fistula when the hoop is in a deployed state. In certain embodiments, the clogging device comprises a balloon.

Aspects of the invention provide a method for treating a fistula by inserting a catheter into the fistula, imaging within the fistula using an imaging device on the catheter, and aspirating the fistula with the catheter. The catheter has an imaging device disposed at a distal portion of the catheter and a clogging device deployable from the catheter. The method optionally includes deploying the clogging device to clog the fistula and then aspirating fluid from the clogged fistula. Where the catheter includes an inner shaft slidably disposed within a lumen of the catheter, the method may include translating the inner shaft relative to the catheter to deploy the clogging device to clog the fistula. In certain embodiments, the catheter includes an aspiration lumen extending parallel to the lumen. A suction device connected to a proximal portion of the aspiration lumen can be used to aspirate fluid from the fistula through the aspiration lumen. Fluid may be aspirated from the fistula through an aspiration lumen extending along the catheter and terminating at an aperture at a distal portion of the catheter.

### BRIEF DESCRIPTION OF THE DRAWINGS

FIG. 1 shows an imaging device for aspirating a fistula.

FIG. 2 depicts an aspirating imaging device according to some embodiments.

FIG. 3 shows a distal portion of an imaging device.

FIG. 4 shows a guidewire extending from a guidewire lumen.

FIG. 5 shows a catheter for intravascular use.

FIG. 6 illustrates the B-mode lateral plane.

FIG. 7 shows production of a C-mode image.

FIG. 8 shows an ultrasonic imaging catheter.

FIG. 9 depicts an ultrasound processing system.

FIG. 10 shows the markers on an aspiration catheter.

FIG. 11 shows radiopaque markers.

FIG. 12 illustrates a pattern of inked markers.

FIG. 13 illustrates a device for aspirating a fistula.

FIG. 14 depicts deployment of a collapsible hoop version of clogging device.

FIG. 15 shows a clogging device in its fully deployed state.

FIG. 16 gives a view of clogging device in its deployed state.

FIG. 17 illustrates a balloon clogging device.

FIG. 18 diagrams a method for aspirating a fistula.

### DETAILED DESCRIPTION

The invention provides devices and methods for the treatment of fistulas and abscesses. Devices of the invention can include an aspiration catheter with imaging capabilities that can also deploy a blocking device, thereby providing an all-in-one treatment device for treating a fistula or an abscess. In some embodiments, the invention provides a device for aspirating an abscess or fistula that includes a catheter having an extended body with a lumen therein, an imaging device such as an ultrasonic transducer disposed at a distal portion of the catheter, and a clogging device deployable from the catheter.

Devices and methods of the invention may also prove useful in treating surgically formed fistulas such as arteriovenous (AV) fistulas created for hemodialysis. A device of the method may be used within an AV fistula to remove obstructions or thrombus and to obtain a desired patency. The aspiration lumen of the device may be used to drain unwanted material from an AV fistula with the clogging device useful for capturing the material to be drained. A device of the invention may be used to clean wastes from blood within the vasculature or kidney(s), remove blood, measure amounts of fluid removal, other functions, or combinations thereof.

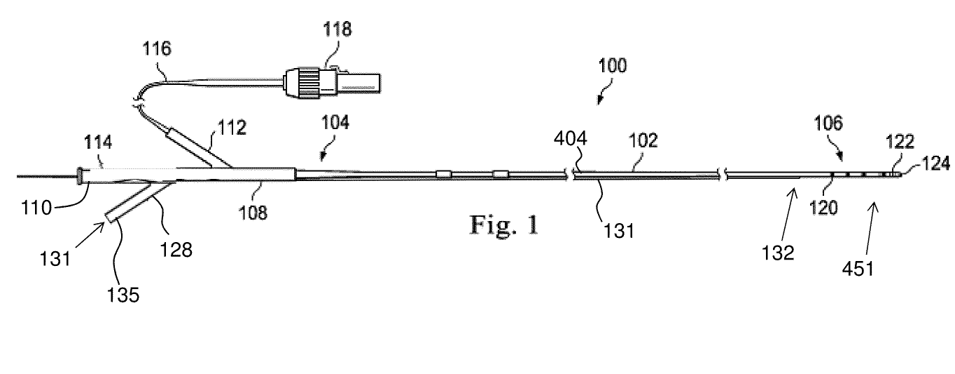

FIG. 1 shows a device 100 for treating a fistula that includes a catheter having an extended body 102 with aspiration lumen 131 and guidewire lumen 404 therein, an imaging device 122 disposed at a distal portion 106 of the catheter, and a clogging device 451 deployable from the catheter. As shown, the imaging device 100 comprises an elongate flexible body 102 having a proximal portion 104 and a distal portion 106. The proximal portion 104 includes an adapter 108 for imaging device 122 and aperture 135 for lumen 131. In the illustrated embodiment, the adapter 108 is multi-forked with extensions 110, 112, and 128. In that regard, extension 110 generally extends along the longitudinal axis of the body 102, while extensions 112 and 128 extends at an oblique angle with respect to the longitudinal axis of the body. Extension 110 is configured to receive a guidewire 114 that is sized and shaped to fit within a guidewire lumen 404 that extends along the length of the body 102 from the proximal portion 104 to the distal portion 106 and defines an opening at the distal end of the imaging device 100. As a result of this arrangement, the imaging device 100 is understood to be what is commonly referred to as an over-the-wire catheter. In some embodiments, the guidewire lumen 404 of the imaging device is centered about the central longitudinal axis of the body 102 (i.e., concentric with aspiration lumen 131). In other embodiments, lumen 404 is offset with respect to the central longitudinal axis of the body 102 (i.e., alongside aspiration lumen 131).

In the illustrated embodiment, extension 112 of adapter 108 is configured to receive communication lines (e.g., electrical, optical, and/or combinations thereof) that are coupled to imaging components positioned within the distal portion 106 of the imaging device 100. In that regard, a cable 116 containing one or more communication lines extends from extension 112 to a connector 118. The connector 118 is configured to interface the imaging device directly or indirectly with one or more of a patient interface module (“PIM”), a processor, a controller, and/or combinations thereof. The particular type of connection depends on the type of imaging components implemented in the imaging device, but generally include one or more of an electrical connection, an optical connection, and/or combinations thereof.

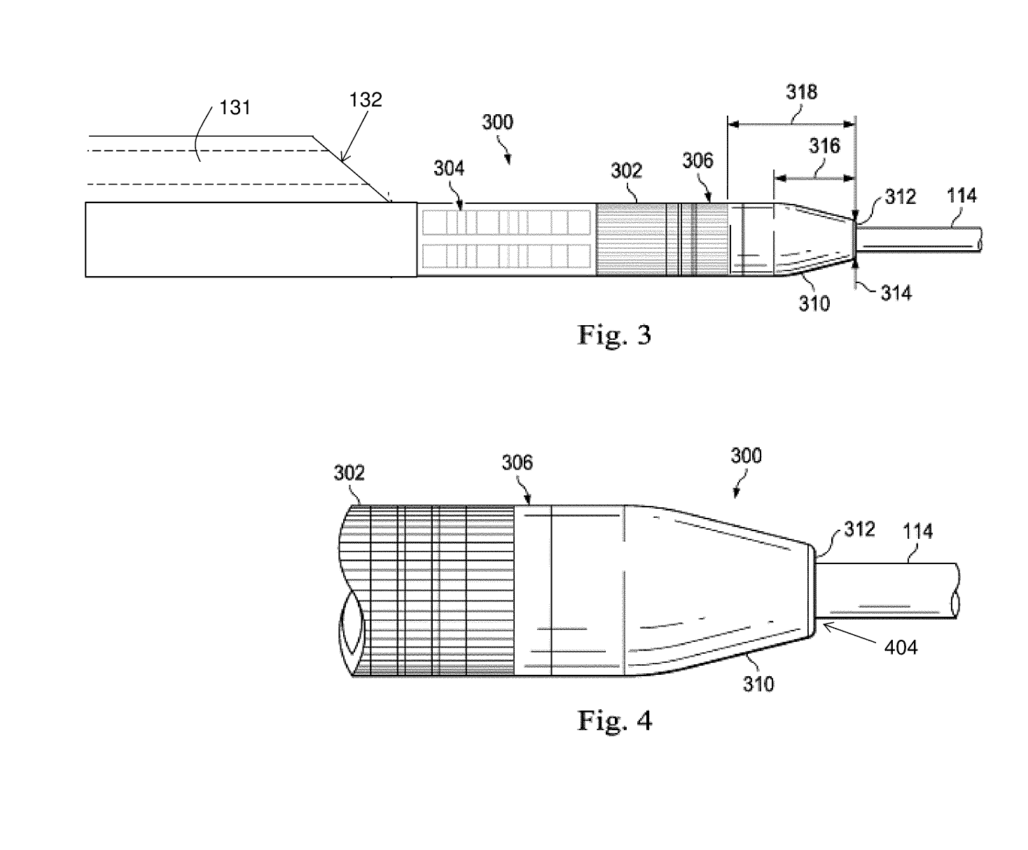

Preferably, catheter device 100 includes an aspiration lumen 131 extending parallel to the guidewire lumen. A suction device may be connected to the aspiration lumen 131. Extension 128 of adapter 108 provides a proximal end of aspiration lumen 131. Aspiration lumen 131 extends through body 102 and a distal end of aspiration lumen 131 is open near distal portion 106 (see, e.g., detail view in FIG. 3). Extension 128 may optionally include a mechanical connector for an aspiration device such as, for example, a Luer lock for a syringe or plunger system.

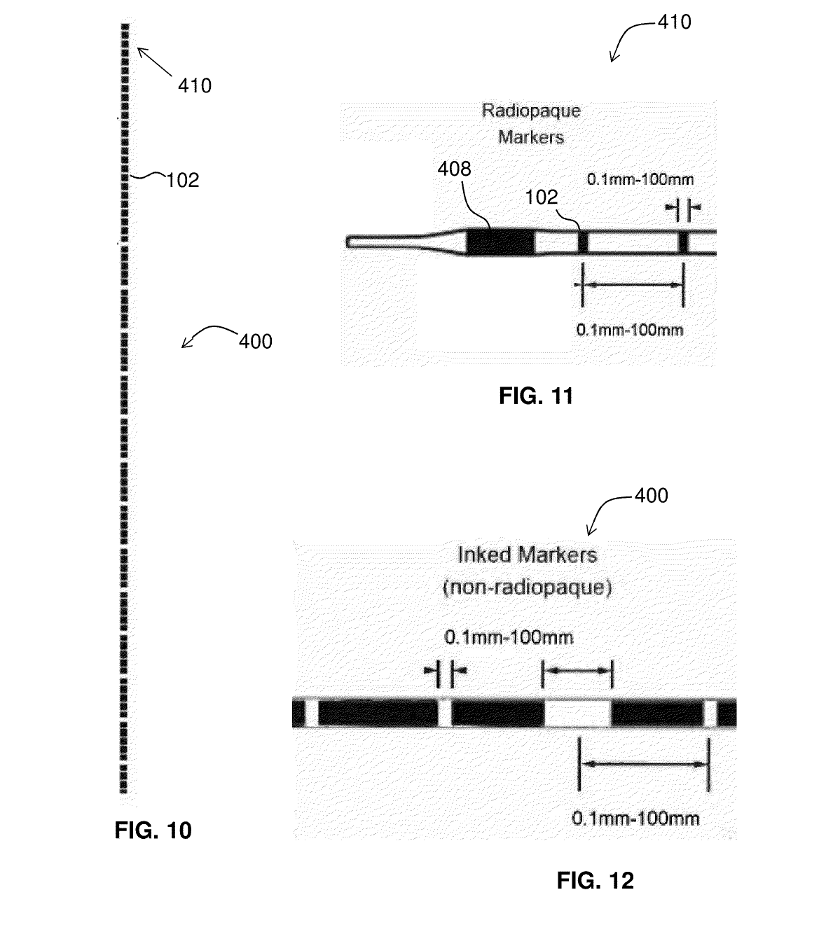

Catheter device 100 includes an imaging device 122 at the distal end. Imaging device 122 may include one or more ultrasound transducer. The ultrasound transducer may use a 64-element cylindrical array that radiates acoustic energy into the surrounding tissue and detects the subsequent echoes. The information from the echoes is used to generate real-time images of the peripheral vessels. Thus, catheter device 100 may be introduced percutaneously or via surgical cutdown into the vascular system, and tracked over 0.035″-0.038″ (0.89-0.97 mm) guide wires. The catheter body may have markers 1 cm apart along the working length (see FIG. 10). A lubricious hydrophilic coating is applied externally to a distal portion of the catheter.

The imaging element 122 may be any type of imaging element suitable for visualizing a fistula. Accordingly, the imaging element may be an ultrasound transducer array (e.g., arrays having 16, 32, 64, or 128 elements are utilized in some embodiments), a single ultrasound transducer, one or more optical coherence tomography (“OCT”) elements (e.g., mirror, reflector, and/or optical fiber), and/or combinations thereof. In that regard, in some embodiments the imaging device 100 is configured to be rotated (either manually by hand or by use of a motor or other rotary device) to obtain images of the vessel. In advanced embodiments, the systems of the invention incorporate focused acoustic computed tomography (FACT), which is described in WO2014/109879, incorporated herein by reference in its entirety.

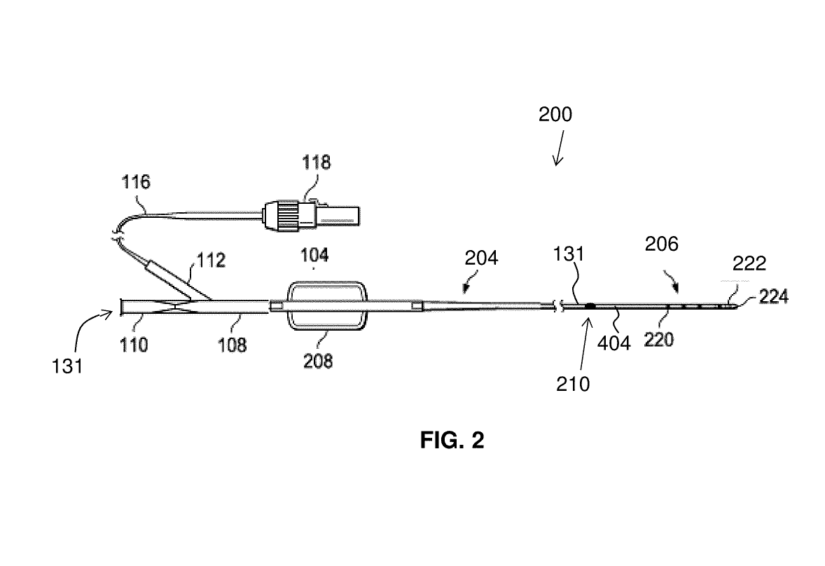

FIG. 2 depicts an imaging device 200 according to another embodiment of the present disclosure. Aspirating imaging device 200 has an elongate flexible body 202 having a proximal portion 204 and a distal portion 206 and defining an aspiration lumen 131. The proximal portion 204 includes a handle 208 for grasping by a user. In the illustrated embodiment, a cable 216 extends from the handle 208 and includes one or more communication lines (e.g., electrical, optical, and/or combinations thereof) that are coupled to imaging components positioned within the distal portion 206 of the imaging device 200. In that regard, a cable 216 containing one or more communication lines extends from handle 208 to a connector 218. The connector 218 is configured to interface the imaging device directly or indirectly with one or more of a patient interface module (“PIM”), a processor, a controller, and/or combinations thereof. The particular type of connection depends on the type of imaging components implemented in the imaging device, but generally include one or more of an electrical connection, an optical connection, and/or combinations thereof.

The body 202 includes an opening 210 that is in communication with a guidewire lumen 404 that extends along the length of the body 202 from the opening 210 to the distal portion 206 and defines an opening at the distal end of the imaging device 200. The opening 210 and the lumen 404 it is in communication with are configured to receive a guidewire. As a result of this arrangement, the imaging device 200 is understood to be what is commonly referred to as a rapid exchange catheter. In some embodiments, the lumen of the imaging device is centered about the central longitudinal axis of the body 202. In other embodiments, the lumen is offset with respect to the central longitudinal axis of the body 202.

The distal portion 206 includes a plurality of markers 220, discussed in greater detail below with respect to FIG. 10.

The imaging element 222 may be any type of imaging element suitable for visualizing a vessel and, in particular, a sever occlusion in a vessel. Accordingly, the imaging element may be an ultrasound transducer array (e.g., arrays having 16, 32, 64, or 128 elements are utilized in some embodiments), a single ultrasound transducer, one or more optical coherence tomography (“OCT”) elements (e.g., mirror, reflector, and/or optical fiber), and/or combinations thereof. In that regard, in some embodiments the imaging device 200 is configured to be rotated (either manually by hand or by use of a motor or other rotary device) to obtain images of the vessel.

FIG. 3 shows a distal portion 300 of an imaging device according to an embodiment of the present disclosure. Distal portion 300 is suitable for use in both over-the-wire catheters (e.g., imaging device 100 of FIG. 1) and rapid exchange catheters (e.g., imaging device 200 of FIG. 2). As shown, the distal portion 300 includes a main body 302 the contains imaging components 304, which may include various electronic, optical, and/or electro-optical components necessary for the particular imaging modality utilized by the imaging device. Distal portion 300 also includes, or is proximal to, aperture 132 of aspiration lumen 131. In the illustrated embodiment, the distal portion 300 of the imaging device is configured for ultrasound imaging and includes an array 306 of ultrasound transducers arranged circumferentially about the distal portion 300 of the imaging device. In that regard, in some embodiments the transducer array 306 and associated components 304 include features as disclosed in U.S. Pat. No. 5,857,974 to Eberle et al. that issued Jan. 12, 1999, U.S. Pat. No. 6,283,921 to Nix et al. that issued on Sep. 4, 2001, U.S. Pat. No. 6,080,109 to Baker et al. that issued on Jun. 27, 2000, U.S. Pat. No. 6,123,673 to Eberle et al. that issued on Sep. 26, 2000, U.S. Pat. No. 6,457,365 to Stephens et al. that issued on Oct. 1, 2002, U.S. Pat. No. 7,762,954 to Nix et al. that issued on Jul. 27, 2010, U.S. Pat. No. 7,846,101 to Eberle et al. that issued on Dec. 7, 2010, and U.S. Patent Application Publication No. 2004/0054287 that published on Mar. 18, 2004, each of which is hereby incorporated by reference in its entirety.

The distal portion 300 also includes a tapered tip portion 310 that extends distally from the main body 302 to the distal end 312. As shown, the tapered tip portion 310 transitions the distal portion 300 from the diameter or thickness 308 to a reduced diameter or thickness 314 at the distal end 312. In some instances, the diameter or thickness 314 is between about 0.30 mm and about 2.5 mm, with some particular embodiments having a diameter or thickness of 0.30 mm (0.012″ or 0.9 French), 0.38 mm (0.015″ or 1.14 French), 0.48 mm (0.019″ or 1.44 French), or otherwise. In that regard, the diameter or thickness 314 is determined based on the desired lumen size for the imaging device in some instances.

FIG. 4 shows a guidewire 114 extending from guidewire lumen 404 of the imaging device such that it extends through an opening in the distal end 312 of the imaging device. In some particular instances, the guidewire 114 has an outer diameter between about 0.28 mm (0.011″ or 0.84 French) and about 0.46 mm (0.018″ or 1.38 French) mm, with some embodiments having an outer diameter of 0.36 mm (0.014″ or 1.07 French). In other instances, the guidewire 114 has outer diameter outside of this range, either larger or smaller. As the distal end 312 of the imaging device defines the opening that receives the guidewire, the diameter or thickness 314 is between 0.28 mm (0.011″ or 0.84 French) and about 0.5 mm (0.020″ or 1.5 French) in some embodiments. In that regard, it is understood that the distal end 312 of the imaging device will necessarily have a slightly larger diameter or thickness than that of the guidewire 114 such that the guidewire can be received therein. However, in some instances the diameter or thickness 314 of the distal end 312 of the imaging device is within 0.03 mm (0.001″ or 0.09 French) or less of the outer diameter of the guidewire. In other instances, the diameter or thickness 314 of the distal end 312 of the imaging device is within 0.5 mm (0.020″ or 1.5 French) or less of the outer diameter of the guidewire.

As shown, the tapered tip portion 310 of the imaging device extends proximal of the distal end 312 by a distance 316. In that regard, the distance 316 is less than 5 mm in some embodiments. Further, the distance 316 is less than 4 mm, less than 3 mm, less than 2 mm, less than 1 mm, and/or less than 0.5 mm from the distal end 312 of the device in some instances. The distance 316 and the difference between the diameter or thickness 308 of the main body 302 and the diameter or thickness 314 at the distal end 312 determine the slope of the outer surface defined by the tapered tip portion 310. In that regard, in some embodiments the tapered tip portion 310 includes a constant taper between the diameter or thickness 308 of the main body 302 at the proximal end of the tapered tip portion and the diameter or thickness 314 at the distal end 312 of the tapered tip portion. In other instances, the tapered tip portion 310 includes a variable taper between the diameter or thickness 308 of the main body 302 at the proximal end of the tapered tip portion and the diameter or thickness 314 at the distal end 312 of the tapered tip portion. For example, in some instances the degree of taper decreases as the tapered tip portion 310 extends distally towards the distal end 312.

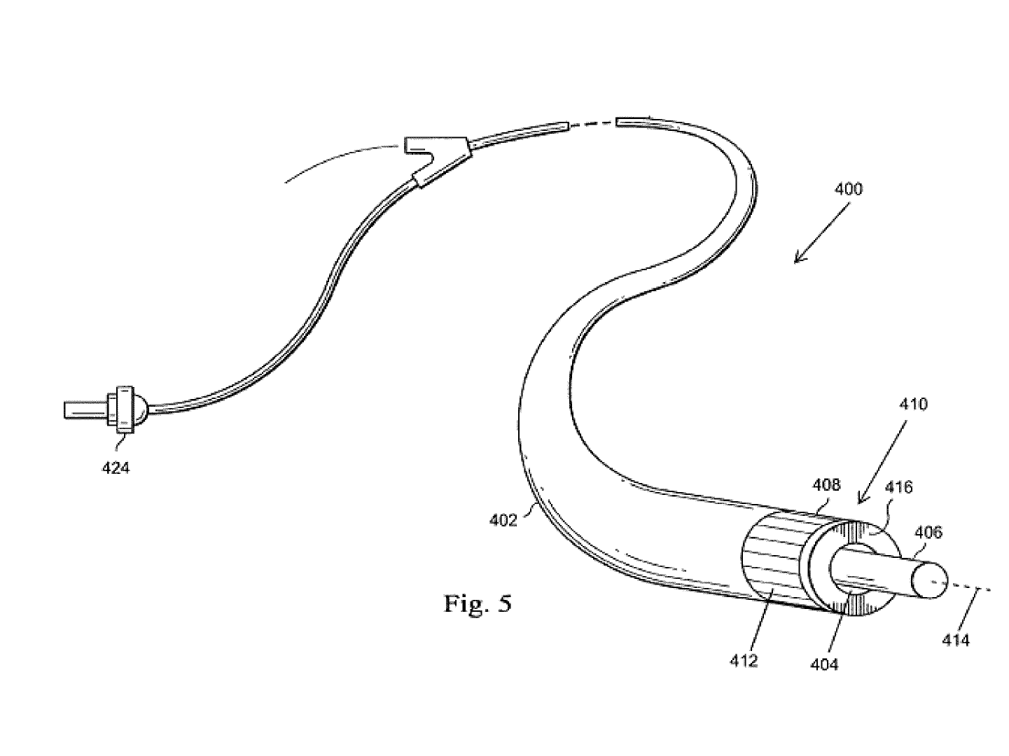

Referring now to FIG. 5, there is shown a catheter 400 for intravascular use, which may be similar to either of imaging devices 100 and 200 discussed above. In that regard, this catheter has an elongated flexible body 402 with an axially extending lumen 404 through which a guide wire 406, fluids, and/or various therapeutic devices or other instruments can be passed. Lumen 404 may be used for aspiration of a fistula or a separate, parallel lumen 131 may be included. The present disclosure is not, however, limited to use with the illustrated catheter arrangements, and it can be utilized with any suitable catheter, guide wire, probe, etc. An ultrasonic imaging transducer assembly 408 is provided at the distal portion 410 of the catheter, with a connector 424 located at the proximal end of the catheter. This transducer 408 comprises a plurality of transducer elements 412 that are preferably arranged in a cylindrical array centered about the longitudinal axis 414 of the catheter for transmitting and receiving ultrasonic energy. The transducer elements 412 are mounted on a cylindrical substrate 416 which, in the embodiment illustrated, consists of a flexible circuit material that has been rolled into the form of a tube. A transducer backing material with the proper acoustical properties surrounds the transducer elements 412.

Each of the transducer elements 412 comprises an elongated body of PZT or other suitable piezoelectric material. The elements extend longitudinally on the cylindrical substrate and parallel to the axis of the catheter. Each element has a rectangular cross-section, with a generally flat surface at the distal end thereof. The transducer elements are piezoelectric material poled in one direction along their entire length. In some embodiments, a transversely extending notch of generally triangular cross-section is formed in each of the transducer elements. The notch opens through the inner surface of the transducer element and extends almost all the way through to the outer surface. Preferably, the notch has a vertical sidewall on the distal side and an inclined sidewall on the proximal side. The vertical wall is perpendicular to the longitudinal axis of the catheter, and the inclined wall is inclined at an angle on the order of 60 degrees to the axis. The notch, which exists in all the array transducer elements, can be filled with a stable non-conductive material. An example of a material that can be used to fill notch is a non-conductive epoxy having low acoustic impedance. Although not the preferred material, conductive materials having low acoustic impedance may also be used to fill notch. If a conductive material is used as the notch filler, it could avoid having to metalize the top portion to interconnect both portions of the transducer elements as required if a nonconductive material is utilized. Conductive materials are not the preferred notch filler given that they have an effect on the E-fields generated by the transducer elements.

In the preferred embodiment, the transducer array provides for a forward looking elevation aperture for 10 mega Hertz (MHz) ultrasound transmit and receive, and a side looking elevation aperture for 20 MHz ultrasound transmit and receive. Other frequencies and/or frequency combinations can be used depending on the particular design requirements or intended uses for the imaging device. The transducer array is manufactured by electrically and mechanically bonding a poled, metalized block of the piezoelectric material to the flexible circuit substrate with the substrate in its unrolled or flat condition. The transducer block exists as a piezoelectric material in a poled state where the thickness-axis poling is generally uniform in distribution and in the same axis throughout the entire block of material. If included, a notch is then formed across the entire piezoelectric block, e.g. by cutting it with a dicing saw. Each of the individual notches is filled with a material such as plastic and a metallization is applied to the top of the notch to form a continuous transducer inner electrode with metallization. The block is then cut lengthwise to form the individual elements that are isolated from each other both electrically and mechanically, with kerfs formed between the elements. Cable wire attachment terminals are provided on the substrate that allow small cables that are electrically connected to an external ultrasound system to connect with the transducer assembly in order to control the transducers.

Integrated circuits are installed on the substrate and the substrate is then rolled into its cylindrical shape, with the transducer elements on the inner side of the cylinder. The sleeve of radiopaque material is mounted on the core, the core is positioned within the cylinder, and the acoustic absorbing material is introduced into the volume between the core and the transducer elements. In the event that a radiopaque marker is not required for a particular application, it can be omitted. The transducer elements 412 can be operated to preferentially transmit and receive ultrasonic energy in either a thickness extensional (TE) mode (k33 operation) or a length extensional (LE) mode (k31 operation). The frequency of excitation for the TE mode is determined by the thickness of the transducer elements in the radial direction, and the frequency for the LE mode is determined by the length of the body between distal end surface and the vertical wall of notch. The thickness TE mode is resonant at a frequency whose half wavelength in the piezoelectric material is equal to the thickness of the element. And the LE mode is resonant at a frequency whose half wavelength in the piezoelectric material is equal to the distance between the distal end and the notch. Each transducer element is capable of individually operating to transmit and receive ultrasound energy in either mode, with the selection of the desired mode (i.e. “side”, or “forward”) being dependent upon; a) an electronically selected frequency band of interest, b) a transducer design that spatially isolates the echo beam patterns between the two modes, and c) image plane specific beam-forming weights and delays for a particular desired image plane to reconstruct using synthetic aperture beam-forming techniques, where echo timing incoherence between the “side” and “forward” beam patterns will help maintain modal isolation.

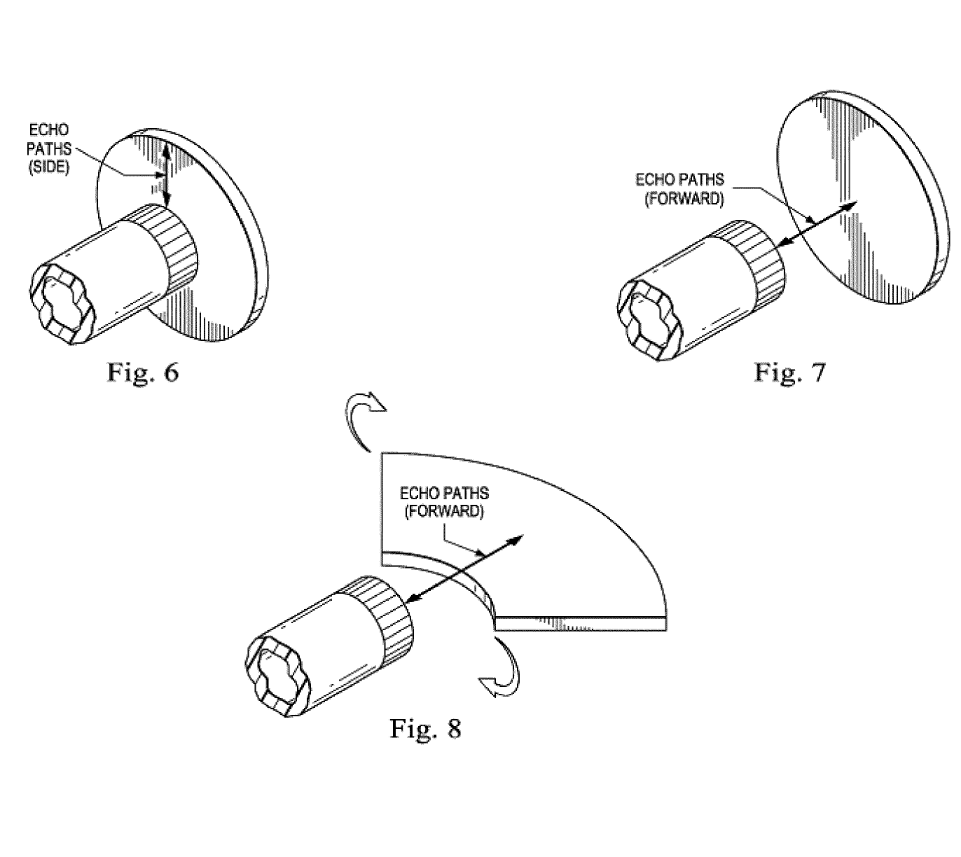

FIGS. 6-8 show various imaging planes that are utilized in some embodiments of the devices and methods of the present disclosure. Some of the ultrasonic imaging catheters of the present disclosure are configured to be “side looking” devices that produce B-mode images in a plane that is perpendicular to the longitudinal axis of the catheter and passes through the transducer.

FIG. 6 illustrates the B-mode lateral plane that is perpendicular to the longitudinal axis of the catheter and passes through the transducer.

FIG. 7 shows production of a C-mode image. Some of the ultrasonic imaging catheters of the present disclosure are configured to be “forward looking” devices that produce a C-mode image plane that is perpendicular to the axis of the catheter and spaced distally from the transducer array.

FIG. 8 shows an ultrasonic imaging catheter of the present disclosure that is configured to be “forward looking” and produce a B-mode image in a plane that extends in a forward direction from the transducer and parallel to the axis of the catheter. That imaging plane is referred to as the B-mode forward plane. Forward viewing devices can be particularly advantageous in some fistulas as they allow the physician to see aspects of the fistula in front of the catheter. Finally, some of the ultrasonic imaging catheters of the present disclosure are configured to transition between two or more of the imaging planes shown in FIGS. 6-8. The following discusses ways these multiple modes of imaging can be implemented. It is understood that some embodiments of the present disclosure implement only a single one of these imaging modes. Further, it is understood that any suitable operating frequencies may be utilized for the different imaging modes, including frequencies between 10 MHz and 80 MHz, including without limitation 10 MHz, 20 MHz, 40 MHz, and 80 MHz. The forward-looking imaging modes described below preferably utilize a 20 MHz operating frequency in some instances.

A piezoelectric transducer, when properly excited, will perform a translation of electrical energy to mechanical energy, and as well, mechanical to electrical. The effectiveness of these translations depends largely on the fundamental transduction efficiency of the transducer assembly taken as a whole. The transducer is a three dimensional electromechanical device though, and as such is always capable of some degree of electromechanical coupling in all possible resonate modes, with one or several modes dominating. Generally an imaging transducer design seeks to create a single dominate mode of electromechanical coupling, suppressing all other coupling modes as “spurious.” The common method used to accomplish a transducer design with a single dominate mode of electromechanical coupling usually rests in the creation of a single, efficient mechanical coupling “port” to the medium outside of the transducer. The single port is created by mounting the transducer such that the most efficient resonant mode of transducer operation faces that mechanical coupling port, with all other modes suppressed by means of mechanical dispersion attained by transducer dimensional control and dampening materials.

In the design of the present disclosure, the transducer design utilizes the fact that a transducer can be effective in two principal electromechanical coupling modes, each mode using a different frequency of operation, acoustic “port”, and electro-mechanical coupling efficiency. One port is the “side looking” port that is used in the cross-sectional view image (as shown in FIG. 6). The other port is the “end” or “forward looking” port of the array (as shown in FIGS. 7 and 8).

The present disclosure allows the two electromechanical coupling modes (i.e. “side” and “forward”) to be always active, without any mechanical switching necessary to choose one mode exclusive of the other. This design also assures that echoes of any image target in the “side looking” plane (see FIG. 6) do not interfere with the target reconstruction in the “forward looking” planes (see FIGS. 7 and 8), and reciprocally, image targets from the “forward looking” do not interfere with the target reconstruction in the “side looking” planes. In accordance with the disclosure, the design methods listed below are used to maintain sufficient isolation between the two modes of operation.

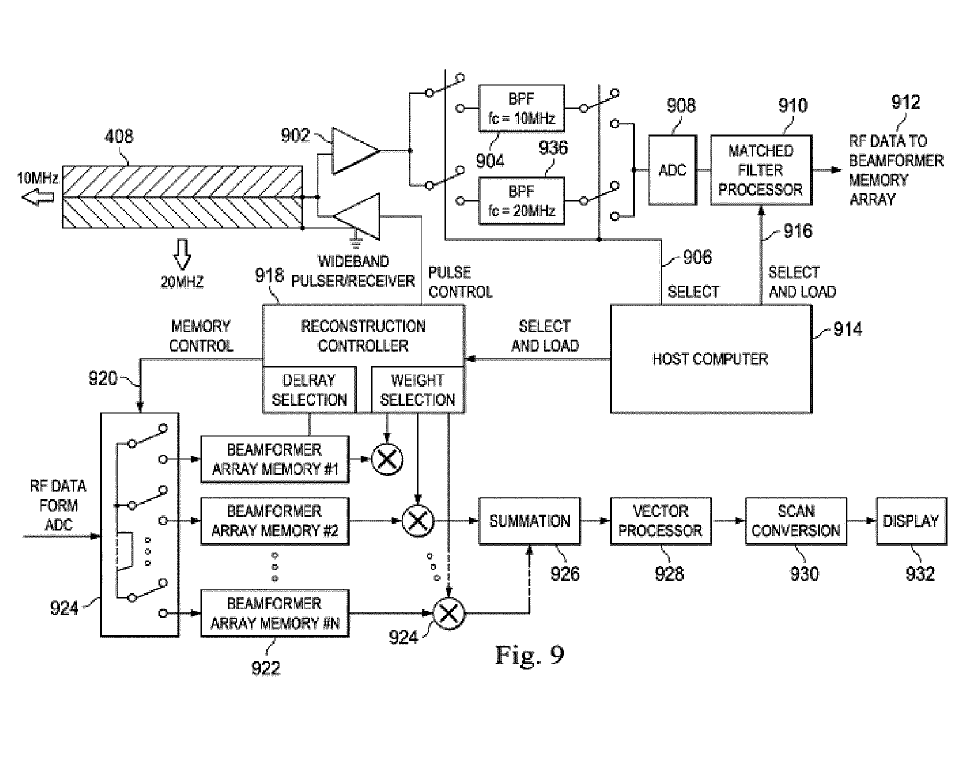

FIG. 9 depicts host ultrasound processing system that controls the ultrasound array 408 element selection and stepping process whereby a single element 412 or multiple elements will transmit and the same or other elements will receive the return echo information. The elements in the array that participate in a given aperture will be sampled sequentially so that all essential cross product transmit-receive terms needed in the beam forming sum are obtained.

The host processing system or computer 914 and reconstruction controller 918 will control the transmit pulse timing provided to wideband receiver 902, the use of any matched filter 910 via control line 916 to perform echo pulse compression. The echo band pass filter (BPF) processing paths in the system are selected using control signal 906 to select between either the 10 MHz 904 or 20 MHz 936 center frequency BPF paths. The amplified and processed analog echo information is digitized using ADC 908 with enough bits to preserve the dynamic range of the echo signals, and passed to the beam-former processing section via signal 912. The beam former section under the control of reconstruction controller 918 uses stored echo data from all the transmit-receive element pairs that exist in an aperture of interest. As the element echo sampling continues sequentially around the circular array, all element group apertures are “reconstructed” using well known synthetic aperture reconstruction techniques to form beam-formed vectors of weighted and summed echo data that radially emanate from the catheter surface using beam-former memory array 922, devices 924 and summation unit 926. Memory control signal 920 controls switch bank 924 which selects which memory array to store the incoming data.

The vector echo data is processed through envelope detection of the echo data and rejection of the RF carrier using vector processor 928. Finally a process of coordinate conversion is done to map the radial vector lines of echo data to raster scan data using scan converter 930 for video display using display 932. Further discussion of imaging catheters suitable for use with the invention may be found in U.S. Pat. 6,457,365 to Stephens; U.S. Pub. 2013/0150716 to Stigall; and U.S. Pub. 2007/0167804 to Park, the contents of which are incorporated by reference.

As described above, the invention provides an aspirating IVUS guided catheter with a clogging device. The device can be used to verify placement, fluid volume, and length of a fistula. The aspirating apparatus is used to verify the type, length and location of the fistula within the body by the use of IVUS. Aspiration catheters are described in U.S. Pat. 8,398,591 to Mas; and U.S. Pub. 2012/0016344 to Kusakabe, the contents of which are incorporated by reference.

To determine the classification of the fistula, a clogging device may be deployed internally to close the fistula so the user can determine the volume of fluid within. IVUS will guide the user to the defined destination to perform surgery The device will have a hub-like design proximally to allow positive/negative pressure (e.g., through attachment of a suction device) thus allowing the abscess to be drained. Where abscesses (a cavity filled with pus) form due to an infection caused by bullet wounds, splinters or other foreign materials, drainage of the abscess may be important. An incision may be made under the skin near the infected area to drain the infection. After draining the pus, an abnormal tube (fistula) may form under the skin and connect to the infected glands of the abscess. Pain, redness, swelling, fatigue, fever and the possibility of external drainage may occur. Devices of the invention may be inserted within the fistula and used to occlude it or alter the surrounding tissue to help close the fistula. Fistulas may be classified based on anatomic (suggests cause and predicts closure), physiologic (predicts morality and metabolic derangements), or etiologic (predicts closure rate) criteria. These classifications—well as type, length and location—help doctors determine methods of treatment. This invention is intended to shorten procedure time by allowing the user to quickly locate the abscess or fistula with IVUS, deploy a clogging device (if needed), determine the classification by defining the volume of fluid within the fistula, and removing the abscess. Thus, the invention provides an all-in-one system that promotes faster and more efficient procedures by quickly locating infected areas and determining fluid volume for classification by the use of a clogging device. A clogging device may prevent fluid travel to major organs, glands, or body parts. It could also promote faster healing of fistula by forcing it to close. In certain embodiments, the invention includes a device with an imaging transducer, radiopaque markers, linear ink markers, outer flexible sheath with lubricious coating, inner flexible shaft to hold a deployable clogging device, and a flexible tip.

The outer or inner shaft or sheath and tip could be made with or a combination of thermoplastics or polymers such as High Density Polyethylene (HDPE), Low Density Polyethylene (LDPE), 25D-72D Polyether Block Amide (Pebax), Polycarbonate (PC), Ethylene Vinyl Acetate (EVA), Polyvinyl Chloride (PVC),Polyether Ether Ketone (PEEK), etc. The shaft or sheath and tip could be, but are not limited to, a single, double, or triple lumen design. A co-extrusion or a blend of these thermoplastics or polymers could be performed.

FIG. 10 shows a pattern for markers on an aspiration catheter 400 of certain embodiments of the present invention. The catheter preferably includes at least one radiopaque marker near a distal portion 410 of the catheter and at least one inked marker. The catheter body may have markers 1 cm apart along the working length. There may be 25 radiopaque markers on the distal end of the catheter, starting 1 cm from the imaging plane, with the 25th radiopaque marker overlapping the distal-most wide inked marker. Inked markers (non-radiopaque) continue along the shaft, spaced 1 cm apart, middle-to-middle, with wider marks indicating 5 cm intervals.

FIG. 11 shows radiopaque markers 120 according to certain embodiments. The distal portion 410 may include a plurality of markers 120. Markers 120 are visible using non-invasive imaging techniques (e.g., fluoroscopy, x-ray, CT scan, etc.) to track the location of the distal portion 106 of the imaging device 100 within a patient. Accordingly, in some instances the markers 120 are radiopaque bands extending around the circumference of the body 102. Further, the markers 120 are positioned at known, fixed distances from an imaging element 122 and/or the distal end 124 of the imaging device 100 in some instances. While the distal portion 106 has been illustrated and described as having a plurality (two or more) of markers 120, in other embodiments the distal portion 106 includes one marker or no markers. Further, in some embodiments, one or more components associated with the imaging element 122 can be utilized as a marker to provide a reference of the position of the distal portion 106 of the imaging device 100.

The radiopaque markers are designed to allow visual exposure under fluoroscopy. Materials for the markers could be, but are not limited to, platinum, gold, palladium, rhenium, iridium, ruthenium, rhodium, etc. They will be placed on the outer, inner part of the device or imbedded such that they can be easily identified internally of the body. The markers could be round-cut hypotubes (marker bands) or a coil (spring). The first or last marker could be aligned with the imaging plane of the transducer to allow the user to know exact placement of the device. Following the first band, the second, third, etc. will be separated 0.1 mm-100 mm allowing the user to identify the distance from the transducer. The bands allow the user to measure the length of the fistula.

FIG. 12 illustrates a pattern of inked markers. Ink markers enable the user to see how far they are inside the patient. Knowing this distance, volume of fluid and the length of the fistula will give the user an approximation of how long to aspirate.

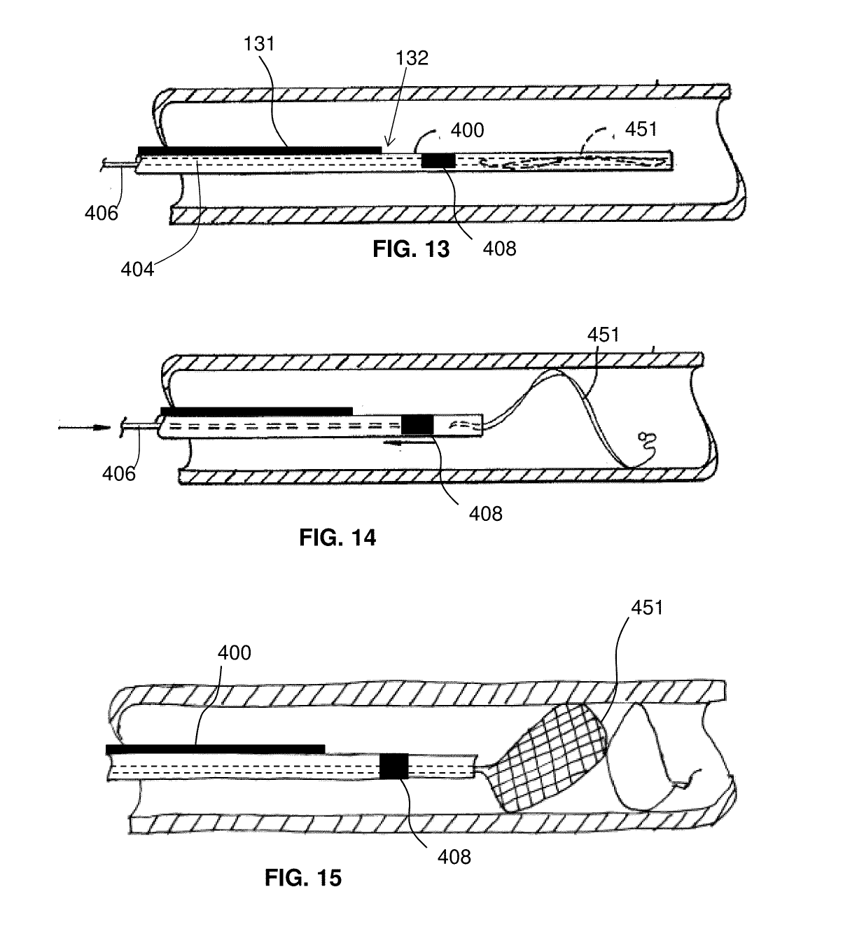

FIG. 13 illustrates a device for aspirating a fistula. The device includes a catheter 400 with an extended body that includes a guidewire lumen 404 and an aspiration lumen 131. Clogging device 451 is included, connected to guidewire 406 (or “inner shaft”), extending through guidewire lumen 404. Imaging transducer 408 is disposed on a distal portion of catheter 400. Aspiration lumen 131 preferably terminates at an aperture 132 for removing fluid or other material from a fistula. In some embodiments, clogging device 451 uses a collapsible hoop that suspends a web to clog the fistula when the hoop is in a deployed state.

FIG. 14 depicts deployment of a collapsible hoop version of clogging device 451. As guidewire 406 is translated through catheter 400, clogging device 451 is extended beyond the distal end of catheter 400, at which point it expands (e.g., due to spring-like pressure from its own structural members). Preferably, clogging device 451 is attached to the inner shaft (i.e., guidewire 406) when in an un-deployed state, and the inner shaft is slidably disposed within the lumen 404.

FIG. 15 shows clogging device 451 in its fully deployed state. The outer members (e.g., nitinol, stainless steel, stiff plastic, or other such material) have expanded, and the preferably non-porous web extends across the fistula. The web may be any suitable material such as plastic, cellophane, PEEK, etc.

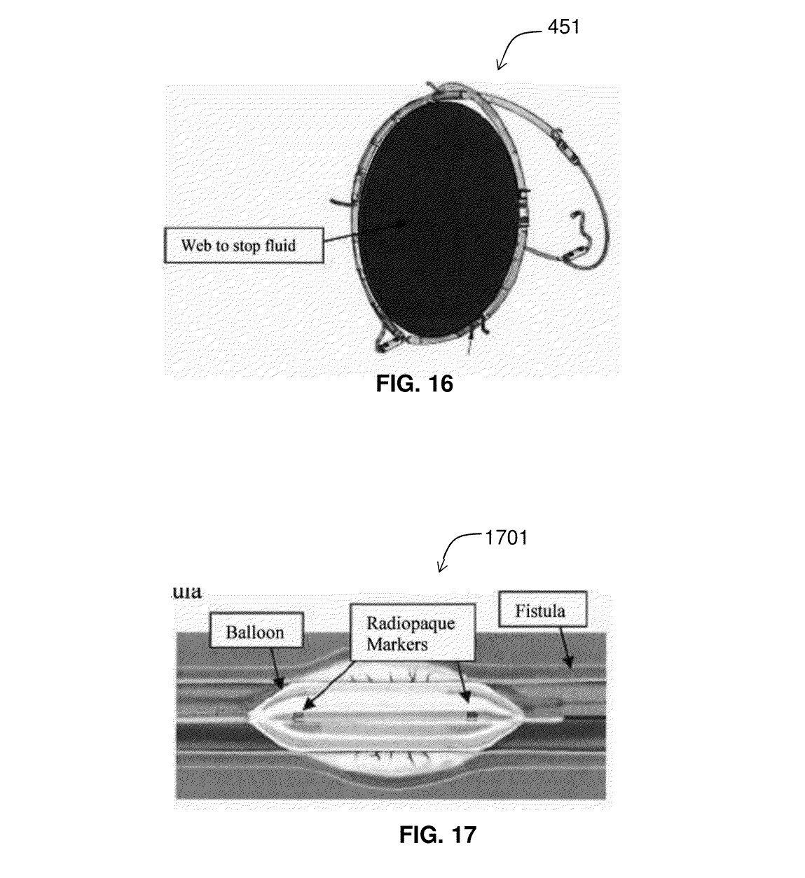

FIG. 16 gives a view of clogging device 451 in its deployed state. Clogging device 451 may be made using techniques described in U.S. Pub. 2008/0147111 to Johnson.

FIG. 17 illustrates an embodiment in which clogging device 451 comprises a balloon 1701. Any suitable balloon can be used. Balloon 1701 may include any suitable material. Generally, balloon 1701 will include a flexible, inelastic material designed to expand. By this type of expansion, a balloon may impose pressures of several atmospheres. After the balloon has been expanded, it is then deflated and removed from the patient. Suitable materials may include polyvinyl chloride (PVC), nylon, polyethylene terephthalate (PET), polybutylene terephthalate (PBT) and copolyesters, polyether-polyester block copolymers, polyamides, polyurethane, poly(ether-block-amide) and the like. Balloons are described in U.S. Pat. No. 7,004,963; U.S. Pub. 2012/0071823; U.S. Pat. No. 5,820,594; and U.S. Pub. 2008/0124495, the contents of each of which are incorporated by reference. Balloon catheters are described in U.S. Pat. No. 5,779,731 and U.S. Pat. No. 5,411,016, incorporated by reference. In certain embodiments, balloon 1701 is used as a clogging device by maneuvering it into position within a fistula and then inflating it (e.g., with a dedicated inflation lumen where catheter 400 is a three-lumen catheter).

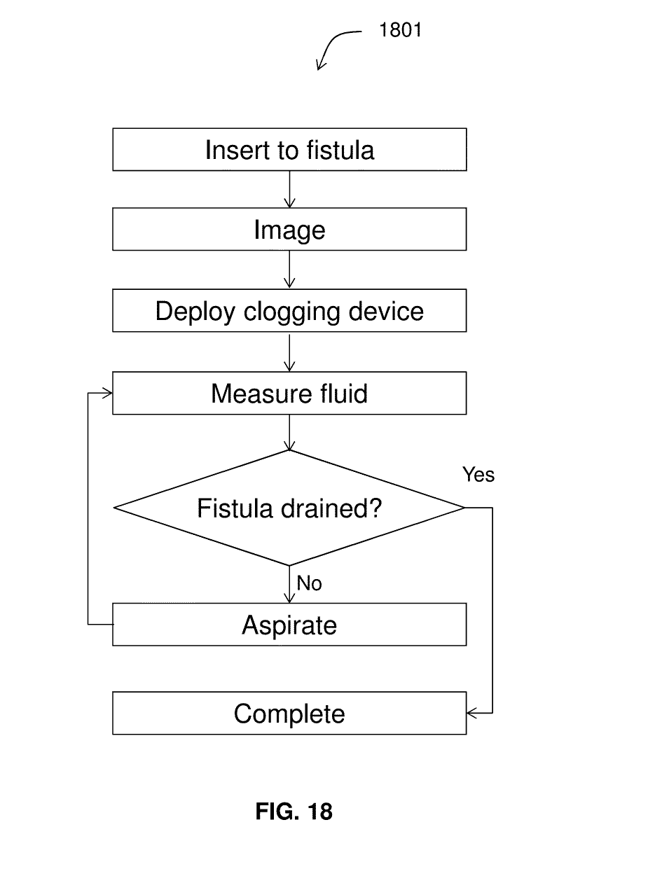

FIG. 18 diagrams a method for aspirating a fistula. The method includes inserting catheter 400 as described herein into a fistula, imaging within the fistula using an imaging device on the catheter, and aspirating the fistula with the catheter. A clogging device 451 may be deployed to clog the fistula and fluid may be aspirated from the clogged fistula. The imaging device may be an ultrasonic transducer. Preferably, catheter 400 includes an inner shaft slidably disposed within the lumen of the catheter. The method can include translating the inner shaft relative to the catheter and deploying the clogging device to clog the fistula. In some embodiments, the catheter further has an aspiration lumen extending parallel to the guidewire lumen, and the method includes using suction from a suction device connected to a proximal portion of the aspiration lumen to aspirate fluid from the fistula through the aspiration lumen. In certain embodiments, the catheter will have at least one radiopaque marker near a distal portion of the catheter and at least one inked marker. The clogging device may use a collapsible hoop that suspends a web to clog the fistula when the hoop is in a deployed state, or may use a balloon. The clogging device could be loaded over the inner shaft and enclosed by the outer shaft. When opening the outer shaft, the clogging device is deployed. This device could promote faster closing of the fistula. It also allows the user to measure the volume of fluid within to classify the infected area.

In some embodiments, the clogging device includes balloon 1701. Using IVUS helps determine the internal diameter of the fistula, which will allow the user to open the balloon to the necessary size. Once the fistula is sealed off, the volume of liquid could be determined. The imaging transducer could be a wired IVUS transducer (similar to Volcano's PV 0.035, EEP, and Revolution catheter) or a wireless/Bluetooth transducer powered by an external device. When imaging inside or outside the body, the image is displayed on a monitor or a set of monitors to allow the user to see what the device is imaging. This allows the user to accurately place the clogging device, measure the length of the fistula, and determine the volume of fluid and type of fistula.

In various embodiments, devices of the invention may include one or more of an imaging transducer to verify location, length and type of fistula; a clogging device to stop abscess drainage; a clogging device to help heal fistula; radiopaque or ink markers to measure length of fistula and to verify length within body; hydrophilic coating for lubricious properties to help aid device in body and around tortuous paths; and a lumen to aspirate fluid out of the fistula. Additionally or alternatively, a device of the invention may include a pressure or velocity sensor to determine, for example, a fluid pressure or to determine a velocity of fluid (e.g., by Doppler flow methods, known in the art). Use of pressure or flow sensors can aid in calculating or determining fractional flow reserve (FFR) or coronary flow reserve (CFR), provided for in certain embodiments. Additional discussion of fistula treatment can be found in U.S. Pub. 2009/0069843 to Agnew; U.S. Pat. No. 7,645,229 to Armstrong; U.S. Pat. No. 6,428,498 to Uflacker; U.S. Pub. 2012/0071838 to Fojtik; and U.S. Pub. 2007/0161963 to Smalling. Material and discussion may also be found in U.S. Pat. No. 8,177,809 to Mavani; U.S. Pub. 2013/0158594 to Carrison; U.S. Pub. 2011/0282334 to Groenhoff, the contents of each of which are incorporated by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A medical device comprising:
a catheter configured for insertion into a fistula, the catheter comprising an aspiration lumen extending therethrough for aspirating material from within a body of a patient;
an imaging device disposed at a distal portion of the catheter for imaging within the fistula; and
a clogging device deployable from the catheter to block drainage from an abscess into the fistula.

2. The device of claim 1, wherein the imaging device comprises an ultrasonic transducer.

3. The device of claim 1, further comprising an inner shaft slidably disposed within a second lumen of the catheter.

4. The device of claim 3, wherein the clogging device is attached to the inner shaft when in an un-deployed state.

5. The device of claim 1, further comprising a suction device connected to the aspiration lumen.

6. The device of claim 1, further comprising one or more conductors extending from the imaging device along a length of the catheter to a connection plug at a proximal end of the catheter, the connection plug configured to be attached to an imaging system instrument.

7. The device of claim 1, further comprising at least one radiopaque marker near a distal portion of the catheter and at least one inked marker.

8. The device of claim 1, wherein the clogging device comprises a collapsible hoop that suspends a non-porous web to clog the fistula when the hoop is in a deployed state.

9. The device of claim 1, wherein the clogging device comprises a balloon.

10. The device of claim 1, further comprising a guidewire lumen.

11. A method for treating a fistula, the method comprising:
inserting a catheter into the fistula, the catheter comprising an imaging device disposed at a distal portion of the catheter and a clogging device deployable from the catheter;
imaging within the fistula using the imaging device; and
aspirating the fistula with the catheter.

12. The method of claim 11, further comprising deploying the clogging device to clog the fistula and then aspirating fluid from the clogged fistula.

13. The method of claim 11, wherein the imaging device comprises an ultrasonic transducer.

14. The method of claim 11, wherein the catheter further comprises an inner shaft slidably disposed within a lumen of the catheter.

15. The method of claim 14, further comprising translating the inner shaft relative to the catheter and deploying the clogging device to clog the fistula.

16. The method of claim 15, wherein the catheter comprises an aspiration lumen extending parallel to the lumen, the method further comprising using suction from a suction device connected to a proximal portion of the aspiration lumen to aspirate fluid from the fistula through the aspiration lumen.

17. The method of claim 11, wherein the catheter further comprises at least one radiopaque marker near a distal portion of the catheter and at least one inked marker.

18. The method of claim 11, wherein the clogging device comprises a collapsible hoop that suspends a non-porous web to clog the fistula when the hoop is in a deployed state.

19. The method of claim 11, wherein the clogging device comprises a balloon.

20. The method of claim 11, further comprising aspirating fluid from the fistula through an aspiration lumen extending along the catheter and terminating at an aperture at a distal portion of the catheter.
